(19) United States
(12) Patent Application Publication
(10) Pub. No.: US 2024/0209118 A1
(43) Pub. Date: Jun. 27, 2024

(54) POLYPEPTIDE LINKER FOR PREPARING MULTISPECIFIC ANTIBODIES

(71) Applicant: BIOMUNEX PHARMACEUTICALS, PARIS (FR)

(72) Inventors: EUGENE ZHUKOVSKY, BETHEL, CT (US); OLIVIER LEGER, SAINT-SIXT (FR)

(21) Appl. No.: 18/589,695

(22) Filed: Feb. 28, 2024

Related U.S. Application Data
(63) Continuation of application No. 16/476,624, filed on Jul. 9, 2019, now Pat. No. 11,958,913.

(30) Foreign Application Priority Data
Jan. 9, 2017 (EP) 17305022.0

(51) Int. Cl. C07K 16/46; C07K 16/28; C07K 16/32

(52) U.S. Cl. CPC C07K 16/468; C07K 16/2827; C07K 16/2863; C07K 16/2896; C07K 16/32; C07K 2317/14; C07K 2317/24; C07K 2317/31; C07K 2317/41; C07K 2317/522; C07K 2317/524; C07K 2317/526; C07K 2317/53; C07K 2317/55

(57) ABSTRACT

The present invention relates to a mutant polypeptide linker for preparing multispecific antibodies, multispecific antibodies, and methods for producing multispecific antibodies.

### CROSS-REFERENCE TO RELATED APPLICATIONS

This application is a continuation of U.S. Ser. No. 16/476,624, filed Jul. 9, 2019, now allowed, which is the U.S. national stage application of International Patent Application No. PCT/EP2018/050481, filed Jan. 9, 2018.

The Sequence Listing for this application is labeled “Seq-List.xml” which was created on Aug. 30, 2022, and is 29,796 bytes. The entire content of the sequence listing is incorporated herein by reference in its entirety.

The present invention relates to multispecific antibodies with improved properties, useful in medical field.

### BACKGROUND OF THE INVENTION

Therapeutic fusion proteins have become an important modality in drug development. Frequently, peptide linkers are used to construct multi-domain proteins from different functional protein modules. The resulting multidomain proteins are designed to bind to the target cognate to the individual modules (or simultaneously exert the biological function of the individual modules) in order to either enhance the biological effects associated with the isolated single domains or to produce novel biological activities unattainable by the isolated single domains. There are numerous examples of molecules that utilize peptide linkers: single chain variable domains of antibodies (scFv), immune-cytokines (cytokine-antibody fusions), bispecific antibodies (BsAb) etc. The choice of linker(s) for a specific fusion protein is dictated by considerations such as: 1) whether the linker(s) require(s) flexibility to afford folding of various domains into a particular tertiary structure (e.g. scFv-based antibodies), 2) whether the linker(s) require rigidity in order to provide a necessary separation between protein domains, or 3) whether the linker(s) must be cleavable in order to permit the separation of domains in vivo to produce the desired activity (Xiaoying Chen, et al, Adv Drug Deliv Rev. 2013. 65(10): 1357-1369). The choice of linkers can be critical since inappropriate linkers may reduce or eliminate the desired activity of the fusion protein (Yumi Maeda, et al, Anal. Biochem. 1997. 249(2): 147-152).

Various linker sequences have been identified for use in the construction of fusion proteins (Richard George and Jaap Heringa, Protein Engineering. 2003. 15(11): 871-879; Xiaoying Chen et al, Adv Drug Deliv Rev. 2013. 65(10): 1357-1369). There are also various available databases that have compiled linker sequences employed in the construction of fusion proteins: 1) SynLinker compiled by the National University of Singapore (synlinker.syncti.org), and 2) The International Genetically Engineered Machine Competition (parts.igem.org/Protein_domains/Linker); Centre for Integrative Bioinformatics at Vrije Universiteit Amsterdam (ibi.vu.nl/programs/linkerdbwww).

International patent application WO2013/005194 discloses multispecific antibodies constructed with linkers designed from the IgG1 hinge sequence, followed by the N-terminal end of the IgG1 CH2 domain sequence followed by the 8 amino acid semi-rigid linker sequence from the central part of the IgA1 hinge. This semi-rigid linker, comprising the central part of the IgA1 hinge, is important to afford for a sufficient separation of both Fab domains of the antibodies, in order to avoid steric hindrance from the C-terminal end of the exterior Fab1 impacted on the antigen-binding paratope of the interior Fc-proximal Fab2.

However, the inventors have identified that the presence of a large number of glycoforms would not make it so easy to produce consistent preparations of such multispecific antibodies required for the development of therapeutics. Additionally, the characterization of such preparations would also be quite complex, which would make comparison of the different manufactured batches laborious.

### SUMMARY OF THE INVENTION

The inventors have then realized that this could be improved, by redesigning the linker, especially by eliminating glycosylation sites from the linker.

The invention thus provides a linker polypeptide which comprises or consists of amino acid sequence EPKX₁CDKX₂HX₃X₄PPX₅PAPELLGGPX₆X₇PPX₈PX₉PX₁₀GG (SEQ ID NO: 1), wherein X₁, X₂, X₃, X₄, X₅, X₆, X₇, X₈, X₉, X₁₀, identical or different, are any amino acid as defined in herein; with the proviso that the polypeptide does not comprise nor consist of sequences

(SEQ ID NO: 5)
EPKSCDKTHTCPPCPAPELLGGPSTPPTPSPSGG
or
(SEQ ID NO: 6)
EPKSCDKTHTSPPSPAPELLGGPSTPPTPSPSGG.

Such polypeptide is useful as a linker in fusion proteins, more particularly multispecific, in particular bispecific, antibodies.

A subject of the invention is thus a multispecific antigen-binding fragment comprising at least two Fab fragments with different CH1 and CL domains, wherein each Fab fragment recognizes a different epitope of interest, and said Fab fragments are tandemly arranged in any order, the C-terminal end of the CH1 domain of a first Fab fragment being linked to the N-terminal end of the VH domain of the following Fab fragment through a polypeptide linker, characterized in that the polypeptide linker sequence comprises or consists of amino acid sequence EPKX1 CDKX2HX3X4PPX5PAPELLGGPX6X7PPX8PX9PX10GG (SEQ ID NO: 1), wherein X1, X2, X3, X4, X5, X6, X7, X8, X9, X10, identical or different, are any amino acid; with the proviso that the linker sequence does not comprise nor consist of sequences

(SEQ ID NO: 5)
EPKSCDKTHTCPPCPAPELLGGPSTPPTPSPSGG
or
(SEQ ID NO: 6)
EPKSCDKTHTSPPSPAPELLGGPSTPPTPSPSGG.

It is further provided a multispecific antibody having two identical antigen-binding arms, each consisting of a multispecific antigen-binding fragment as defined herein.

In a preferred embodiment, it is provided a multispecific antibody that has an immunoglobulin-like structure, comprising:
two identical antigen-binding arms each consisting of a multispecific antigen-binding fragment as defined herein;
the dimerized CH2 and CH3 domains of an immunoglobulin;
the hinge region of an IgA, IgG, or IgD, linking the C-terminal ends of CH1 domains of the antigen-binding arms to the N-terminal ends of the CH2 domains.

The invention thus more particularly provides a multispecific, preferably a bispecific antibody, comprising two heavy chains and four light chains, wherein each heavy chain comprises
a. a Fc region of an immunoglobulin comprising Hinge-CH2-CH3 domains,
b. which Fc region is linked to Fab heavy chain CH1-VH of antibody 1 (Ab1) by said Hinge domain,
c. which in turn is linked to Fab heavy chain CH1-VH of antibody 2 (Ab2), by a polypeptide linker sequence, wherein the polypeptide linker sequence links the N-terminus of said Fab heavy chain VH domain of Ab1 with the C-terminus of said CH1 domain of Ab2, and the four light chains comprise light chains of Ab1 and light chains of Ab2 associated with their cognate heavy chain domains; characterized in that the polypeptide linker sequence comprises or consists of amino acid sequence EPKX₁CDKX₂HX₃X₄PPX₅PAPELLGGPX₆X₇PPX₈PX₉PX₁₀GG (SEQ ID NO: 1), wherein X₁, X₂, X₃, X₄, X₅, X₆, X₇, X₈, X₉, X₁₀, identical or different, are any amino acids; with the proviso that the linker sequence does not comprise nor consist of sequences

(SEQ ID NO: 5)
EPKSCDKTHTCPPCPAPELLGGPSTPPTPSPSGG
or
(SEQ ID NO: 6)
EPKSCDKTHTSPPSPAPELLGGPSTPPTPSPSGG.

In a particular embodiment, the polypeptide linker sequence comprises or consists of a sequence selected from the group consisting of

(SEQ ID NO: 2)
EPKSCDKTHTSPPAPAPELLGGPGGPPGPGPGGG;
(SEQ ID NO: 3)
EPKSCDKTHTSPPAPAPELLGGPAAPPAPAPAGG;
and
(SEQ ID NO: 4)
EPKSCDKTHTSPPAPAPELLGGPAAPPGPAPGGG.

A further subject of the invention is a polypeptide which comprises, preferably consists of, a heavy chain of the multispecific antigen-binding fragment, or multispecific, preferably bispecific antibody, as defined herein.

The invention further provides a polynucleotide comprising a sequence encoding such polypeptide.

A host cell transfected with an expression vector comprising said polynucleotide is also part of the invention.

A further subject of the invention is a method for producing a multispecific antibody, preferably a bispecific antibody, as described herein, said method comprising the following steps: a) culturing in suitable medium and culture conditions a host cell expressing an antibody heavy chain as defined herein, and an antibody light chain as defined herein; and b) recovering said produced antibodies from the culture medium or from said cultured cells.

### LEGENDS TO THE FIGURES

FIG. 1 is a schematic drawing of a full-length BiXAb bispecific antibody of the invention.

FIG. 2 is a schematic drawing of a bispecific construct that contains only two Fab domains connected by a linker without a Fc-domain (Fab-Fab).

FIG. 3A shows the Size Exclusion chromatography analysis of BiXAb2b.

FIG. 3B shows the Size Exclusion chromatography analysis of BiXAb3b.

FIG. 3C shows the Size Exclusion chromatography analysis of Fab-Fab3b.

FIG. 4A shows the MS spectrum of the LC-MS analysis of Fab-Fab3a.

FIG. 4B shows the MS spectrum of the LC-MS analysis of Fab-Fab3b.

FIG. 5A shows the MS spectrum of the LC-MS analysis of BiXAb3a.

FIG. 5B shows the MS spectrum of the LC-MS analysis of BiXAb3b.

### DETAILED DESCRIPTION OF THE INVENTION

### Definitions

The basic structure of a naturally occurring antibody molecule is a Y-shaped tetrameric quaternary structure consisting of two identical heavy chains and two identical light chains, held together by non-covalent interactions and by inter-chain disulfide bonds.

In mammalian species, there are five types of heavy chains: α, δ, ε, γ, and μ, which determine the class (isotype) of immunoglobulin: IgA, IgD, IgE, IgG, and IgM, respectively. The heavy chain N-terminal variable domain (VH) is followed by a constant region, containing three domains (numbered CH1, CH2, and CH3 from the N-terminus to the C-terminus) in γ, α, and δ heavy chains, while the constant regions of μ and ε heavy chains are composed of four domains (numbered CH1, CH2, CH3 and CH4 from the N-terminus to the C-terminus). The CH1 and CH2 domains of IgA, IgG, and IgD are separated by a flexible hinge, which varies in length between the different classes and in the case of IgA and IgG, between the different subtypes: IgG1, IgG2, IgG3, and IgG4 have respectively hinges of 15, 12, 62 (or 77), and 12 amino acids, and IgA1 and IgA2 have respectively hinges of 20 and 7 amino acids.

There are two types of light chains: λ and κ, which can associate with any of the heavy chain isotypes, but are both of the same type in a given antibody molecule. Both light chains appear to be functionally identical. Their N-terminal variable domain (VL) is followed by a constant region consisting of a single domain termed CL.

The heavy and light chains pair by protein/protein interactions between the CH1 and CL domains, and between the VH and VL domains, and the two heavy chains associate by protein/protein interactions between their CH3 domains.

The antigen-binding regions correspond to the arms of the Y-shaped structure, which consist each of the complete light chain paired with the VH and CH1 domains of the heavy chain, and are called the Fab fragments (for Fragment antigen binding). Fab fragments were first generated from native immunoglobulin molecules by papain digestion which cleaves the antibody molecule in the hinge region, on the amino-terminal side of the interchain disulfide bonds, thus releasing two identical antigen-binding arms. Other proteases such as pepsin, also cleave the antibody molecule in the hinge region, but on the carboxy-terminal side of the interchain disulfide bonds, releasing fragments consisting of two identical Fab fragments and remaining linked through disulfide bonds; reduction of disulfide bonds in the F(ab′)2 fragments generate Fab′ fragments.

The part of the antigen-binding region corresponding to the VH and VL domains is called the Fv fragment (for Fragment variable); it contains the CDRs (complementarity determining regions), which form the antigen-binding site (also termed paratope).

The effector region of the antibody which is responsible for its binding to effector molecules on immune cells, corresponds to the stem of the Y-shaped structure, and contains the paired CH2 and CH3 domains of the heavy chain (or the CH2, CH3 and CH4 domains, depending on the class of antibody), and is called the Fc (for Fragment crystallisable) region.

Due to the identity of the two heavy chains and the two light chains, naturally occurring antibody molecules have two identical antigen-binding sites and thus bind simultaneously to two identical epitopes.

In the context of the invention, the “multispecific antigen-binding fragment” is defined herein as a molecule having two or more antigen-binding regions, each recognizing a different epitope. The different epitopes can be borne by a same antigenic molecule or by different antigenic molecules. The term “recognizing” or “recognizes” means that the fragment specifically binds a target antigen.

An antibody “specifically binds” to a target antigen if it binds with greater affinity, avidity, more readily, and/or with greater duration than it binds to other substances. “Specific binding” or “preferential binding” does not necessarily require (although it can include) exclusive binding. Generally, but not necessarily, reference to binding means preferential binding.

The terms “subject,” “individual,” and “patient” are used interchangeably herein and refer to a mammal being assessed for treatment and/or being treated. Subjects may be human, but also include other mammals, particularly those mammals useful as laboratory models for human disease, e.g. mouse, rat, rabbit, dog, etc.

The term “treatment” or “treating” refers to an action, application or therapy, wherein a subject, including a human being, is subjected to medical aid with the purpose of improving the subject's condition, directly or indirectly. Particularly, the term refers to reducing incidence, or alleviating symptoms, eliminating recurrence, preventing recurrence, preventing incidence, improving symptoms, improving prognosis or combination thereof in some embodiments. The skilled artisan would understand that treatment does not necessarily result in the complete absence or removal of symptoms. For example, with respect to cancer, “treatment” or “treating” may refer to slowing neoplastic or malignant cell growth, proliferation, or metastasis, preventing or delaying the development of neoplastic or malignant cell growth, proliferation, or metastasis, or some combination thereof.

### Design of the Multispecific Antibodies

It is herein provided multispecific antigen-binding fragment(s) and multispecific antibody constructs, comprising said fragments, wherein each multispecific antigen-binding fragment consists essentially of tandemly arranged Fab fragments, separated by the linker of the invention.

Such fragments and constructs preferably comprise chains from human immunoglobulins, preferably IgG, still preferably IgG1.

In case of a multispecific antigen-binding fragment comprising more than two different Fab fragments, the polypeptide linkers separating the Fab fragments can be identical or different.

According to a preferred embodiment of a multispecific antibody of the invention, it has two identical antigen-binding arms, each consisting of a multispecific antigen-binding fragment as defined above. The antigen-binding arms can be linked together in diverse ways, depending on the intended use for the antibody.

If one wishes to obtain an antibody without Fc-mediated effects, the antibody will comprise no Fc region. In this case, the two antigen-binding arms can be linked together for instance:
by homodimerization of the antigen-binding arms through the inter-chain disulfide bonds provided by the polypeptide linker(s) separating the Fab fragments; and/or
through the addition at the C-terminal end of each antigen-binding arm, of a polypeptide extension containing cysteine residues allowing the formation of inter-chain disulfide bonds, and homodimerization of said polypeptide extension resulting in a hinge-like structure; byway of non-limitating examples, said polypeptide extension may be for instance a hinge sequence of an IgG1, IgG2 or IgG3;
through a semi-rigid linker joining the C-terminal ends of the heavy chains of the two antigen-binding arms to form a single polypeptide chain and maintaining said antigen-binding arms at a sufficient distance between each other.

Alternatively, if effector functions such as CDC, ADCC or ADP are desired, a multispecific antibody of the invention can further comprise a Fc domain providing these effector functions. The choice of the Fc domain will depend on the type of desired effector functions.

In this case, a multispecific antibody of the invention has an immunoglobulin-like structure, comprising:
two identical multispecific antigen-binding arms as defined above;
the dimerized CH2 and CH3 domains of an immunoglobulin;
either the hinge region of an IgA, IgG, or IgD, linking the C-terminal ends of the CH1 domains of the antigen-binding arms to the N-terminal ends of the CH2 domains, or alternatively, when the CH4 domains that follow the CH3 domains come from an IgM or IgE, the C-terminal ends of the CH1 domains of the antigen-binding arms being in this case can be linked directly to the N-terminal ends of the CH2 domains.

Preferably, the CH2 and CH3 domains, the hinge region and/or the CH4 domains are derived from a same immunoglobulin or from immunoglobulins of the same isotype and subclass as the CH1 domains of the antigen-binding arm.

The CH2, CH3, and optionally CH4 domains, as well as the hinge regions from native immunoglobulins can be used. It is also possible to mutate them, if desired, for instance in order to modulate the effector function of the antibody. In some instances, whole or part of the CH2 or the CH3 domain can be omitted.

The invention more particularly provides bispecific tetravalent antibodies, comprising two binding sites to each of their targets, and a functional Fc domain allowing the activation of effector functions such as antibody-dependent cell-mediated cytotoxicity (ADCC) and phagocytosis.

Such preferred antibodies of the invention are full length antibodies. They preferably comprise heavy chains and light chains from human immunoglobulins, preferably IgG, still preferably IgG1.

The light chains may be lambda or kappa light chains; they preferably are Kappa light chains.

In a preferred embodiment, the linker of the invention links IgG Fab domains in a tetra-Fab bispecific antibody format, the amino acid sequence of which comprises the heavy chain sequences of at least two Fab joined by said polypeptide linker, followed by the native hinge sequence, followed by the IgG Fc sequence, co-expressed with the appropriate IgG light chain sequences.

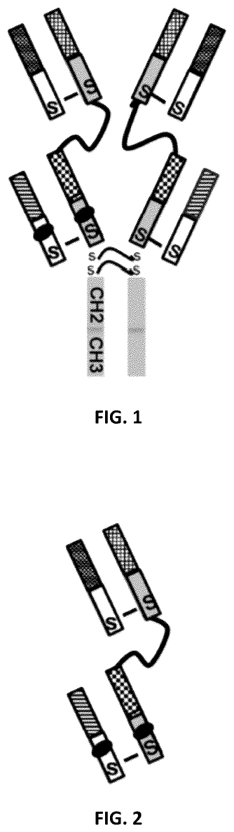

An example of the antibodies of the invention, named BiXAb antibodies, which have an IgG-like structure, is illustrated in FIG. 1. Antibodies designated BiXAb2a, BiXAb2b, BiXAb2c, and BiXAb3b, described below, are particular examples.

The bispecific antibodies of the invention typically comprise
a continuous heavy chain constructed of an Fc (Hinge-CH2-CH3),
followed by antibody 1 Fab heavy chain (CH1-VH) and the successive Fab heavy chain (CH1-VH) of antibody 2, the latter joined by a polypeptide linker sequence of the invention,
and during protein expression the resulting heavy chain assembles into dimers while the co-expressed antibody 1 and antibody 2 light chains (VL-CL) associate with their cognate heavy chains to form the final tandem Fab-Fab-Fc molecule, the antibody 1 (Ab1) and the antibody 2 (Ab2) being different.

In a preferred embodiment, described are bispecific antibodies, which comprise
two Fab fragments with different CH1 and CL domains consisting of
a) Fab fragment having CH1 and C-Kappa domains derived from a human IgG1/Kappa, and the VH and VL domains of Ab1,
b) Fab fragment having CH1 and C-Kappa domains derived from a human IgG1/Kappa and the VH and VL domains of Ab2,
c) a mutated light chain CL constant domain which is derived from human Kappa constant domain,
d) a mutated heavy chain CH1 constant domain
the Fab fragments being tandemly arranged in the following order
   the C-terminal end of the CH1 domain of Ab1 Fab fragment being linked to the N-terminal end of the VH domain of Ab2 Fab fragment through a polypeptide linker,
   the hinge region of a human IgG1 linking the C-terminal ends of CH1 domain of Ab2 fragment to the N-terminal of the CH2 domain,
   the dimerized CH2 and CH3 domains of a human IgG1.

Ab1 and Ab2 may be any antibody of interest, especially any antibody of therapeutic interest.

In a particular embodiment, Ab1 and Ab2, being different, independently are selected from the group consisting of an anti-EGFR antibody and an anti-HER2/neu antibody. In a preferred embodiment, Ab1 and Ab2, being different, independently are selected from the group consisting of cetuximab or a mutated derivative thereof, on the one hand, and trastuzumab, or a mutated derivative thereof, on the other hand.

In another particular embodiment, Ab1 and Ab2, being different, independently are selected from the group consisting of an anti-CD38 antibody and an anti-PD-L1 antibody.

Such antibodies are useful as a medicament, more particularly in treating a cancer.

Throughout the present description, amino acid sequences are defined according to Kabat et al, Sequences of Proteins of Immunological Interest, 5th Ed. Public Health Service, National Institutes of Health, Bethesda, Md. (1991).

Another example of the constructs of the invention, which is a multispecific antigen-binding fragment Fab-Fab, which does not contain the Fc domain, is illustrated in FIG. 2. A particular example, designated Fab-Fab3a, is described below.

Such Fab-Fab constructs typically comprise two different Fab domains. Such antibodies possess only one Fab domain each that binds to antigen 1 and to antigen 2.

They possess the same Light Chains as in the corresponding BiXAb antibodies; however, the Heavy Chain of Fab-Fabs is shortened in such a fashion so that their most C-terminal residue is Cysteine-220 (in EU numbering).

### Design of the Linkers

The polypeptide linker sequence according to the invention comprises or consists of amino acid sequence EPKX₁CDKX₂HX₃X₄PPX₅PAPELLGGPX₆X₇PPX₈PX₉PX₁₀GG (SEQ ID NO: 1), wherein X₁, X₂, X₃, X₄, X₅, X₆, X₇, X₈, X₉, X₁₀, identical or different, are any amino acid; with the proviso that the linker sequence does not comprise nor consist of sequences

(SEQ ID NO: 5)
EPKSCDKTHTCPPCPAPELLGGPSTPPTPSPSGG
or
(SEQ ID NO: 6)
EPKSCDKTHTSPPSPAPELLGGPSTPPTPSPSGG.

The polypeptide linker sequence consists of less than 80 amino acids, preferably less than 60 amino acids, still preferably less than 40 amino acids.

In a particular embodiment, X₁, X₂ and X₃, identical or different, are Threonine (T) or Serine (S).

In another particular embodiment, X₁, X₂ and X₃, identical or different, are any amino acids other than Threonine (T) or Serine (S), preferably wherein X₁, X₂ and X₃, identical or different, are selected from the group consisting of Ala (A), Gly (G), Val (V), Asn (N), Asp (D) and Ile (I), still preferably X₁, X₂ and X₃, identical or different, may be Ala (A) or Gly (G).

Alternatively, X₁, X₂ and X₃, identical or different, may be Leu (L), Glu (E), Gln (Q), Met (M), Lys (K), Arg (R), Phe (F), Tyr (T), His (H), Trp (W), preferably Leu (L), Glu (E), or Gln (Q).

In a particular embodiment, X₄ and X₅, identical or different, are any amino acid selected from the group consisting of Serine (S), Cysteine (C), Alanine (A), and Glycine (G).

In a preferred embodiment, X₄ is Serine (S) or Cysteine (C).

In a preferred aspect, X₅ is Alanine (A) or Cysteine (C).

In a particular embodiment, X₆, X₇, X₈, X₉, X₁₀, identical or different, are any amino acid other than Threonine (T) or Serine (S). Preferably X₆, X₇, X₈, X₉, X₁₀, identical or different, are selected from the group consisting of Ala (A), Gly (G), Val (V), Asn (N), Asp (D) and Ile (I).

Alternatively, X₆, X₇, X₈, X₉, X₁₀, identical or different, may be Leu (L), Glu (E), Gln (Q), Met (M), Lys (K), Arg (R), Phe (F), Tyr (T), His (H), Trp (W), preferably Leu (L), Glu (E), or Gln (Q).

In a preferred embodiment, X₆, X₇, X₈, X₉, X₁₀, identical or different, are selected from the group consisting of Ala (A) and Gly (G).

In still a preferred embodiment, X₆ and X₇ are identical and are preferably selected from the group consisting of Ala (A) and Gly (G).

In a preferred embodiment, the polypeptide linker sequence comprises or consists of sequence SEQ ID NO: 1, wherein
X₁, X₂ and X₃, identical or different, are Threonine (T), Serine (S);
X₄ is Serine (S) or Cysteine (C);
X₅ is Alanine (A) or Cysteine (C);
X₆, X₇, X₈, X₉, X₁₀, identical or different, are selected from the group consisting of Ala (A) and Gly (G).

In a particular embodiment, the polypeptide linker sequence comprises or consists of a sequence selected from the group consisting of

(SEQ ID NO: 2)
EPKSCDKTHTSPPAPAPELLGGPGGPPGPGPGGG;
(SEQ ID NO: 3)
EPKSCDKTHTSPPAPAPELLGGPAAPPAPAPAGG;
and
(SEQ ID NO: 4)
EPKSCDKTHTSPPAPAPELLGGPAAPPGPAPGGG.

In another preferred embodiment, the polypeptide linker sequence comprises or consists of sequence SEQ ID NO: 1, wherein
X₁, X₂ and X₃, identical or different, are Ala (A) or Gly (G);
X₄ is Serine (S) or Cysteine (C);
X₅ is Alanine (A) or Cysteine (C);
X₆, X₇, X₈, X₉, X₁₀, identical or different, are selected from the group consisting of Ala (A) and Gly (G).
   Production of the Antibodies Nucleic acids encoding heavy and light chains of the antibodies of the invention are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Such control sequences include a signal sequence, a promoter, an enhancer, and a transcription termination sequence. Expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences.

In one example, both the heavy and light chain coding sequences (e.g., sequences encoding a VH and a VL, a VH-CH1 or a VL-CL, are included in one expression vector. In another example, each of the heavy and light chains of the antibody is cloned into an individual vector. In the latter case, the expression vectors encoding the heavy and light chains can be co-transfected into one host cell for expression of both chains, which can be assembled to form intact antibodies either in vivo or in vitro.

In a particular embodiment, a host cell is co-transfected with three independent expression vectors, such as plasmids, leading to the coproduction of all three chains (namely the heavy chain HC, and two light chains LC1 and LC2, respectively) and to the secretion of the multispecific antibody.

More especially the three vectors may be advantageously used in a following molecular ratio of 3:2:2 (HC:LC1:LC2).

The recombinant vectors for expression of the antibodies described herein typically contain a nucleic acid encoding the antibody amino acid sequences operably linked to a promoter, either constitutive or inducible. The vectors can be suitable for replication and integration in prokaryotes, eukaryotes, or both. Typical vectors contain transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, i.e., shuttle vectors, and selection markers for both prokaryotic and eukaryotic systems.

Multispecific antibodies as described herein may be produced in prokaryotic or eukaryotic expression systems, such as bacteria, yeast, filamentous fungi, insect, and mammalian cells. It is not necessary that the recombinant antibodies of the invention be glycosylated or expressed in eukaryotic cells; however, expression in mammalian cells is generally preferred. Examples of useful mammalian host cell lines are human embryonic kidney line (293 cells), baby hamster kidney cells (BHK cells), Chinese hamster ovary cells/− or + DHFR (CHO, CHO-S, CHO-DG44, Flp-in CHO cells), African green monkey kidney cells (VERO cells), and human liver cells (Hep G2 cells).

Mammalian tissue cell culture is preferred to express and produce the polypeptides because a number of suitable host cell lines capable of secreting intact immunoglobulins have been developed in the art, and include the CHO cell lines, various Cos cell lines, HeLa cells, preferably myeloma cell lines, or transformed B-cells or hybridomas.

In a most preferred embodiment, the multispecific, preferably bispecific, antibodies of the invention are produced by using a CHO cell line, most advantageously a CHO-S cell line.

Expression vectors for these cells can include expression control sequences, such as an origin of replication, a promoter, and an enhancer, and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like.

The vectors containing the polynucleotide sequences of interest (e.g., the heavy and light chain encoding sequences and expression control sequences) can be transferred into the host cell by well-known methods, which vary depending on the type of cellular host. For example calcium phosphate treatment or electroporation may be used for other cellular hosts. (See generally Sambrook et al., Molecular Cloning: A Laboratory Manual (Cold Spring Harbor Press, 2nd ed., 1989). When heavy and light chains are cloned on separate expression vectors, the vectors are co-transfected to obtain expression and assembly of intact immunoglobulins.

Host cells are transformed or transfected with the vectors (for example, by chemical transfection or electroporation methods) and cultured in conventional nutrient media (or modified as appropriate) for inducing promoters, selecting transformants, or amplifying the genes encoding the desired sequences.

Once expressed, the whole antibodies, their dimers, individual light and heavy chains, or other immunoglobulin forms of the present invention can be further isolated or purified to obtain preparations that are substantially homogeneous for further assays and applications.

Standard protein purification methods known in the art can be used. For example, suitable purification procedures may include fractionation on immunoaffinity or ion-exchange columns, ethanol precipitation, high-performance liquid chromatography (HPLC), sodium dodecyl sulfate polyacrylamide gel electrophoresis (SDS-PAGE), ammonium sulfate precipitation, and gel filtration (see generally Scopes, Protein Purification (Springer-Verlag, N.Y., 1982). Substantially pure immunoglobulins of at least about 90 to 95% homogeneity are preferred, and 98 to 99% or more homogeneity most preferred, for pharmaceutical uses.

In vitro production allows scale-up to give large amounts of the desired multispecific, preferably bispecific, antibodies of the invention. Such methods may employ homogeneous suspension culture, e.g. in an airlift reactor or in a continuous stirrer reactor, or immobilized or entrapped cell culture, e.g. in hollow fibers, microcapsules, on agarose microbeads or ceramic cartridges.

### Therapeutic Applications

A further aspect of the invention is a pharmaceutical composition comprising an antibody according to the invention. Another aspect of the invention is the use of an antibody according to the invention for the manufacture of a pharmaceutical composition. A further aspect of the invention is a method for the manufacture of a pharmaceutical composition comprising an antibody according to the invention.

In another aspect, the present invention provides a composition, e.g. a pharmaceutical composition, containing an antibody as defined herein, formulated together with a pharmaceutical carrier.

A composition of the present invention can be administered by a variety of methods known in the art.

The present invention, thus generally described above, will be understood more readily by reference to the following examples, which are provided by way of illustration and are not intended to be limiting of the instant invention.

### EXAMPLES

### Design

A first bispecific antibody of the invention is the antibody designated BiXAb2a having the following structure:
i) a continuous heavy chain which comprises
   Trastuzumab heavy chain variable region (VH) corresponding to SEQ ID NO: 7
   Wild-type CH1 constant domain (the residue at Kabat position 192 is threonine) from human IgG1 corresponding to SEQ ID NO: 8
      Polypeptide linker joining the 2 Fab heavy chains consisting of

(SEQ ID NO: 2)
EPKSCDKTHTSPPAPAPELLGGPGGPPGPGPGGG;

Cetuximab heavy chain variable region (VH) corresponding to SEQ ID NO: 9
Mutated CH1 constant domain (the residue at Kabat position 192 has been mutated from threonine to glutamic acid) from human IgG1 corresponding to SEQ ID NO: 10
Wild-type Hinge region from human IgG1 corresponding to SEQ ID NO: 11
Wild-type CH2 domain of human IgG1 corresponding to SEQ ID NO: 12
Wild-type CH3 domain of human IgG1 corresponding to SEQ ID NO: 13 So, the bispecific antibody of the invention has a continuous heavy chain (701 residues) of SEQ ID NO: 14
ii) a wild-type trastuzumab light chain which consists of SEQ ID NO: 15
iii) a cetuximab light chain with a mutated constant domain (the residues at Kabat positions Ser 114 and Asn 137 have been mutated to Ala and Lys, respectively) from human Kappa corresponding to SEQ ID NO: 16.

A second bispecific antibody of the invention is the antibody designated BiXAb2b which is comprised of the same sequences, except for the linker, which is EPKSCDKTHTSPPAPAPELLGGPAAPPAPAPAGG (SEQ ID NO: 3).

(SEQ ID NO: 3)
EPKSCDKTHTSPPAPAPELLGGPAAPPAPAPAGG.

A third bispecific antibody of the invention is the antibody designated BiXAb2c which is comprised of the same sequences, except for the linker, which is

(SEQ ID NO: 4)
EPKSCDKTHTSPPAPAPELLGGPAAPPGPAPGGG.

A fourth bispecific antibody of the invention is the antibody designated BiXAb3b having the following structure:
i) a continuous heavy chain which comprises
   Atezolizumab heavy chain variable region (VH) corresponding to SEQ ID NO: 23 (EVQLVESGGGLVQPGGSLRLSCAASGFTFSDSWIHWVRQAPGKGLEWVAWISPYGGST YYADSVKGRFTISADTSKNTAYLQMNSLRAEDTAVYYCARRHWPGGFDYWGQGTLVTVS S)
   Mutated CH1 constant domain (the residue at Kabat position 192 has been mutated from threonine to glutamic acid) from human IgG1 corresponding to SEQ ID NO: 10

Polypeptide linker joining the 2 Fab heavy chains consisting of SEQ ID NO: 3; Daratumumab heavy chain variable region (VH) corresponding to SEQ ID NO: 17 (EVQLLESGGGLVQPGGSLRLSCAVSGFTFNSFAMSWVRQAPGKGLEWVSAISGSGGGT YYADSVKGRFTISRDNSKNTLYLQMNSLRAEDTAVYFCAKDKILWFGEPVFDYWGQGTLVT VSS)
Wild-type CH1 constant domain (the residue at Kabat position 192 is threonine) from human IgG1 corresponding to SEQ ID NO: 8
Wild-type Hinge region from human IgG1 corresponding to SEQ ID NO: 11
Wild-type CH2 domain of human IgG1 corresponding to SEQ ID NO: 12
Wild-type CH3 domain of human IgG1 corresponding to SEQ ID NO: 13

[So the heavy chain of the invention has a continuous heavy chain of SEQ ID NO: 18

EVQLVESGGGLVQPGGSLRLSCAASGFTFSDSWIHwVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVEVPSSSLGTQTYI
CNVNHKPSNTKVDKKVEPKSCDKTHTSPPAPAPELLGGPAAPPAPAPAGG
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK]

ii) an atezolizumab light chain with a mutated constant domain (the residues at Kabat positions Ser 114 and Asn 137 have been mutated to Ala and Lys, respectively) from human Kappa corresponding to SEQ ID NO: 19

DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPAVFIFPPSDEQLKSGTASVVCLLKNFYPREAKVQWKV
DNALQSGNSQESVTEQDSKDSTYSLSSTLTLSKADYEKHKVYACEVTHQG
LSSPVTKSFNRGEC

iii) a wild-type daratumumab light chain which consists of SEQ ID NO: 20

EIVLTQSPATLSLSPGERATLSCRASQSVSSYLAVVYQQKPGQAPRLLIY
DASNRATGIPARFSGSGSGTDFTLTISSLEPEDFAVYYCQQRSNWPPTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQG
LSSPVTKSFNRGEC.

For comparison purposes, an antibody designated BiXAb3a was also produced which differs from the BiXAb3b antibody by the linker which consists of

(SEQ ID NO: 6)
EPKSCDKTHTSPPSPAPELLGGPSTPPTPSPSGG.

Another construct of the invention is designated Fab-Fab3b; it is comprised of the same sequence as BiXAb3b, except that the hinge, CH2 and CH3 domains are missing in the heavy chain. So, Fab-Fab3b has a continuous heavy chain of SEQ ID NO: 21 EVQLVESGGGLVQPGGSLRLSCAASGFTFSDSWIHWVRQAPGKGLEWVAWISPYGGSTY YADSVKGRFTISADTSKNTAYLQMNSLRAEDTAVYYCARRHWPGGFDYWGQGTLVTVSS ASTKGPSVFPLAPSSKSTSGGTAALGCLVKDYFPEPVTVSWNSGALTSGVHTFPAVLQSS GLYSLSSVVEVPSSSLGTQTYICNVNHKPSNTKVDKKVEPKSCDKTHTSPPAPAPELLGGP AAPPAPAPAGGEVQLLESGGGLVQPGGSLRLSCAVSGFTFNSFAMSWVRQAPGKGLEWV SAISGSGGGTYYADSVKGRFTISRDNSKNTLYLQMNSLRAEDTAVYFCAKDKILWFGEPVF DYWGQGTLVTVSSASTKGPSVFPLAPSSKSTSGGTAALGCLVKDYFPEPVTVSWNSGALT SGVHTFPAVLQSSGLYSLSSVVTVPSSSLGTQTYICNVNHKPSNTKVDKKVEPKSC.

For comparison purposes, the construct designated Fab-Fab3a was also produced, that is comprised of the same sequence as BiXAb3a, except that the hinge, CH2 and CH3 domains are missing in the heavy chain.

SEQ ID NO: 7 to 16 are shown below.

SEQ ID NO: 7
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSS
SEQ ID NO: 8
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKV
SEQ ID NO: 9
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSA
SEQ ID NO: 10
ASTKGPSVFPLAPSSKSTSGGTAALGCLVKDYFPEPVTVSWNSGALTSGV
HTFPAVLQSSGLYSLSSVVEVPSSSLGTQTYICNVNHKPSNTKVDKKV
SEQ ID NO: 11
EPKSCDKTHTCPPCP
SEQ ID NO: 12
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK
SEQ ID NO: 13
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK
SEQ ID NO: 14
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTSPPAPAPELLGGPGGPPGPGPG
GGQVQLKQSGPGLVQPSQSLSITCTVSGFSLTNYGVHWVRQSPGKGLEWL
GVIWSGGNTDYNTPFTSRLSINKDNSKSQVFFKMNSLQSNDTAIYYCARA
LTYYDYEFAYWGQGTLVTVSAASTKGPSVFPLAPSSKSTSGGTAALGCLV
KDYFPEPVTVSWNSGALTSGVHTFPAVLQSSGLYSLSSVVEVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
SEQ ID NO: 15
DIQMTQSPSSLSASVGDRVTITCRASQDVNTAVAWYQQKPGKAPKWYSAS
FLYSGVPSRFSGSRSGTDFTLTISSLQPEDFATYYCQQHYTTPPTFGQGT
KVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGEC
SEQ ID NO: 16
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPAVFIFPPSDEQLKSGTASVVCLLKNFYPREAKVQWKV
DNALQSGNSQESVTEQDSKDSTYSLSSTLTLSKADYEKHKVYACEVTHQG
LSSPVTKSFNRGEC

### Gene Synthesis

The amino acid sequences of anti-HER2 (trastuzumab, clone humAb4D5-8) and anti-EGFR (cetuximab) were used to design the DNA sequences after codon optimization for mammalian expression using GeneScript program. For the heavy chain, the DNAs encoding signal peptides, variable region and constant CH1 domain of Fab1 followed the pseudo hinge linker and variable region and constant CH1 domain of Fab2 with flanking sequences for restriction enzyme digestion were synthesized by GeneScript. For the light chain, the DNAs encoding signal peptides and variable and constant Kappa regions were synthesized by GeneScript.

PCR reactions using PfuTurbo Hot Start were carried out to amplify the inserts which were then digested by NotI+ApaI and NotI+HindIII for heavy and light chains, respectively. The double digested heavy chain fragments were ligated with NotI+ApaI treated pcDNA3.1 expression vector (Invitrogen) in which the human IgG1 CH1+hinge+CH2+CH3 domains were already inserted. The double digested light chain fragments were ligated with NotI+HindIII treated pcDNA3.1 expression vector (Invitrogen). Plasmid DNAs were verified by double strand DNA sequencing.

### Expression and Purification

The bispecific antibodies of the invention were produced by means of transient gene expression by co-transfection of 3 genes coded on separate vectors in a 2:3:3=HC:LC1:LC2 molar ratio (1 continuous heavy chain (HC) and 2 light chains (LC)) in CHO-S cells adapted to serum-free medium in suspension (CHO SFM-II medium from Life Technologies™) Typically, for 50 mL medium scale expression testing, a total of 50 μg of plasmid DNAs (25 μg heavy chain 1, 12.5 μg of tratuzumab light chain and 12.5 μg of cetuximab light chain) were mixed in 1.5 mL Eppendorf tube, 1 mL of CHO SFM medium containing 25 μL of 3 mg/mL PEI transfection reagent (POLYPLUS) pH7.0 was added, incubated at RT for 20 min. The mixture of DNA-PEI was loaded into 49 mL of Life Technologies' Invitrogen FreeStyle™ CHO-S cells at 1˜2×10⁶/mL in 125 mL shaking flask. Cells were shaken for 6 more days. The supernatant was harvested by centrifuging cells at 3,000 rpm for 15 min. The expression titer of the BiXAbs in the supernatant was determined using FortéBio's protein A biosensors (Octet@ Systems). The bispecific monoclonal antibody (BiXAb) was then purified on protein A affinity medium using MabSelect SuRe (GE Healthcare Life Sciences). The antibody was eluted from protein A using 0.1 M glycine pH 3.5 with neutralization in 1 M TRIS. The purified antibody in Dulbecco's PBS (Lonza BE17-512Q) was sterile-filtered (0.2 μM sterile filters from Techno Plastic Products AG) and the final concentration determined by OD reading at 280 nm using Eppendorf BioSpectrometer®.

### SDS-PAGE Analysis

Electrophoresis was performed under reducing conditions and non-reducing conditions employing Gel Biorad Stain-Free 4-15% gels and the corresponding running buffer. Samples were prepared by combining the purified BiXAb or Fab-Fab antibodies with 2X SDS sample buffer and heating for 5 min at 95° C. Preparation of reduced samples included the addition of NuPAGE reducing agent prior to heating. The apparent MW was determined using Ladder Precision Plus Protein Unstained Standards (Biorad).

Size Exclusion chromatography analysis Protein aggregation is frequently observed in engineered protein molecules. We performed analytical size exclusion chromatography (SEC) to assay the high molecular weight species content of our antibodies. We employed an SEC-s3000 (300×7.8 mm) column (BioSep) and an Aktapurifier 10 system (GE Healthcare); the assay was conducted at a flow rate of 1 mL/min using PBS buffer pH 7.4.

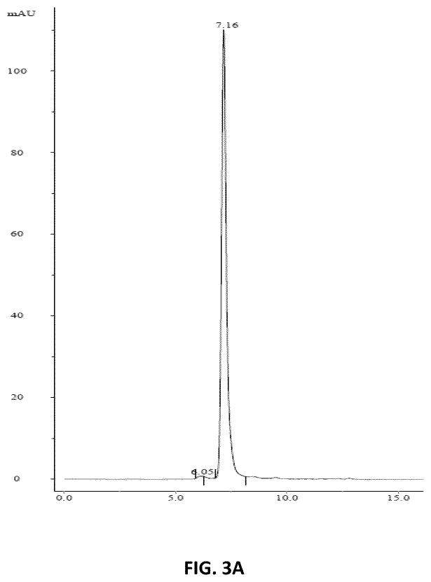

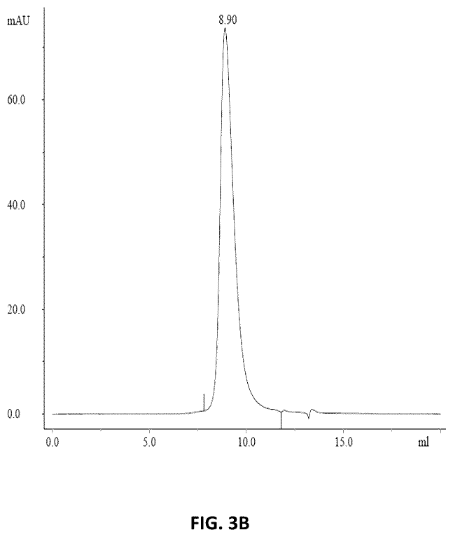

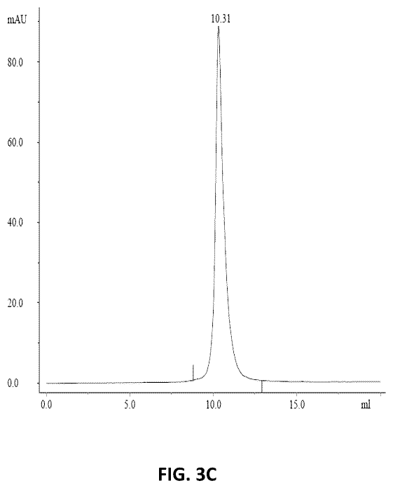

The SEC chromatograms of BiXAb2b (FIG. 3A), BiXAb3a, BiXAb3b (FIG. 3B), Fab-Fab3a, and Fab-Fab3b (FIG. 3C) demonstrated that the main peak corresponded to the expected sizes of the monomeric BiXAb and Fab-Fab antibodies; these peaks represented 99.9-100.0% of the total sample. Thus, we concluded that the antibodies containing the new linker possess no high molecular weight species. The narrow and symmetric shape of the monomeric peaks suggested that all BiXAbs and Fab-Fabs were correctly assembled and represented by a single species.

Characterization of BiXAbs by Differential Scanning Calorimetry Differential Scanning Calorimetry (DSC) was used to test the thermal stability of BiXAb2b. A Microcal™ VP-Capillary DSC system (Malvern Instruments) was used to perform differential scanning calorimetry experiments.

Samples were centrifuged (20,000×g, 5 min, 4° C.), and their protein content was quantitated prior to the DSC analysis using a NANODROP ND-1000 spectrophotometer (THERMO SCIENTIFIC) employing the IgG analysis program. For assay, samples were diluted in PBS to a final concentration of 1 mg/mL.

The pre-equilibration time was 3 min, and the resulting thermograms were acquired between 20 and 110° C. at a scan rate of 60° C./h, a filtering period of 25 see, and medium feedback. Prior to sample analysis, 5 buffer/buffer scans were measured to stabilize the instrument, and a buffer/buffer scan was performed between each protein/buffer scan. The data were fit to a non-2-state unfolding model, with the pre- and post-transition adjusted by subtraction of the baseline.

The DSC results demonstrated that the DSC profile of BiXAb2b exhibited two transitions. The smaller peak had a Cp max of 96 Kcal/mole/° C. and a Tm1 of 71.5° C., corresponding to the unfolding of both CH2 and Fab domains, and the larger peak had a Cp max of 190 Kcal/mole/° C. and a Tm2 of 80.5° C., corresponding to the unfolding of the CH3 domain.

### Liquid Chromatography/Mass Spectroscopy (LC-MS) Analysis

LC-MS/MS data were acquired using a Dionex Ultimate 3000 system coupled to a Q-EXACTIVE mass spectrometer (Thermo Fisher Scientific, Bremen, Germany) and a PROSWIFT RP-4H reverse phase column (250 mm×1 mm; Thermo Fisher). The column oven temperature was set to 65° C. Ten microliters were injected for LC separation. A gradient of mobile phases consisting of LC-MS-grade water with 0.1% formic acid (phase A) and acetonitrile with 0.1% formic acid (phase B) was delivered at a flow rate of 0.2 mL/min (total run time of 20 minutes). Eluted antibody species were introduced into the Q-EXACTIVE instrument by electrospray ionization (ESI), which operated in positive ion mode using full-scan 15,000 resolution. Xcalibur 2.2 software (Thermo Fisher Scientific, Bremen, Germany) was used for instrument control and processing of the data files.

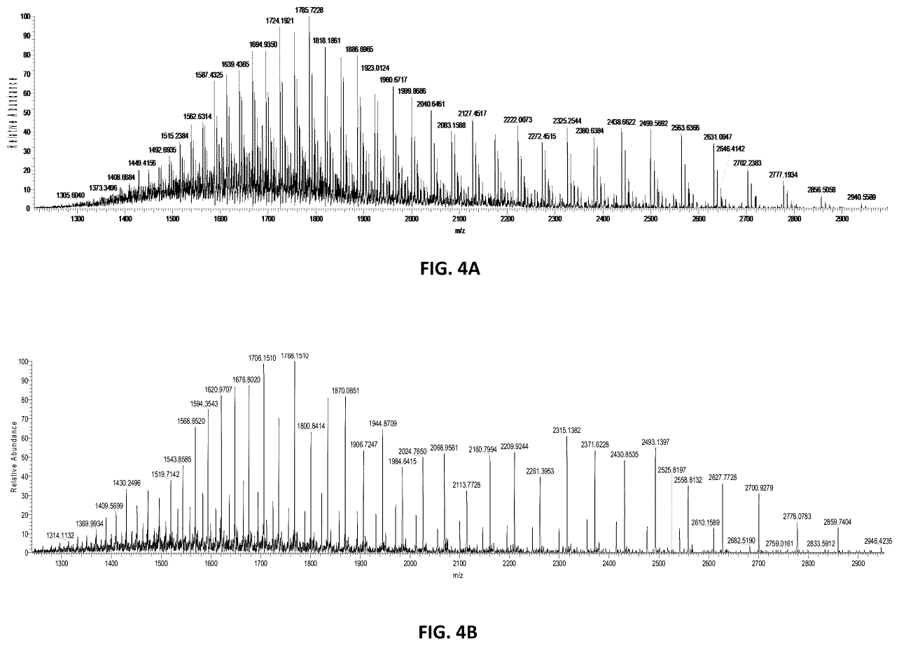

FIGS. 4A and 4B present the LC-MS analysis of Fab-Fab3a (containing a linker with SEQ ID NO: 6) and Fab-Fab3a (containing a linker with SEQ ID NO: 3), respectively. FIG. 4A demonstrates that the LC-MS spectrum of Fab-Fab3a with a linker corresponding to SEQ ID NO: 6 is significantly more heterogeneous than that of the related Fab-Fab3b antibody, which differed only in the composition of the linker (SEQ ID NO: 3). The linker sequence in the Fab-Fab3a antibody contains the PSTPPTPSPS (SEQ ID NO:22) sequence, which is found in the human IgA1 hinge and is known to be subject to O-linked glycosylation at 2 threonine and 2 serine residues. Since glycosylation of these sites is heterogeneous, the product usually possesses multiple glycoforms. whose populations are strongly affected by the expression conditions of the recombinant proteins. The total number of N-linked and O-linked glycosylation sites in Fab-Fab3a is at least 4, explaining the complex MS spectrum observed in FIG. 4A. The sequence of the linker corresponding to SEQ ID NO: 3 was designed in order to reduce heterogeneity due to non-homogeneous O-linked glycosylation. As such, several Serine and Threonine residues in the portion of the linker, whose sequence matched that of the sequence of the hinge of human IgA1 known to undergo O-linked glycosylation, were replaced with Glycine residues; additionally, several other Serine and Threonine residues were also replaced with Glycine in the linker. FIG. 4B shows that the MS spectrum of Fab-Fab3b containing the SEQ ID NO: 3 linker is substantially simplified; this is because the Fab-Fab3b analyte is less complex than the Fab-Fab3a analyte whose spectrum is presented in FIG. 4A. Thus, we concluded that the elimination of 4 O-linked glycosylation sites from the Fab-Fab3a antibody by replacing the linker of SEQ ID NO: 6 with the SEQ ID NO: 3 linker, resulted in a substantially more homogeneous BiXAb preparation.

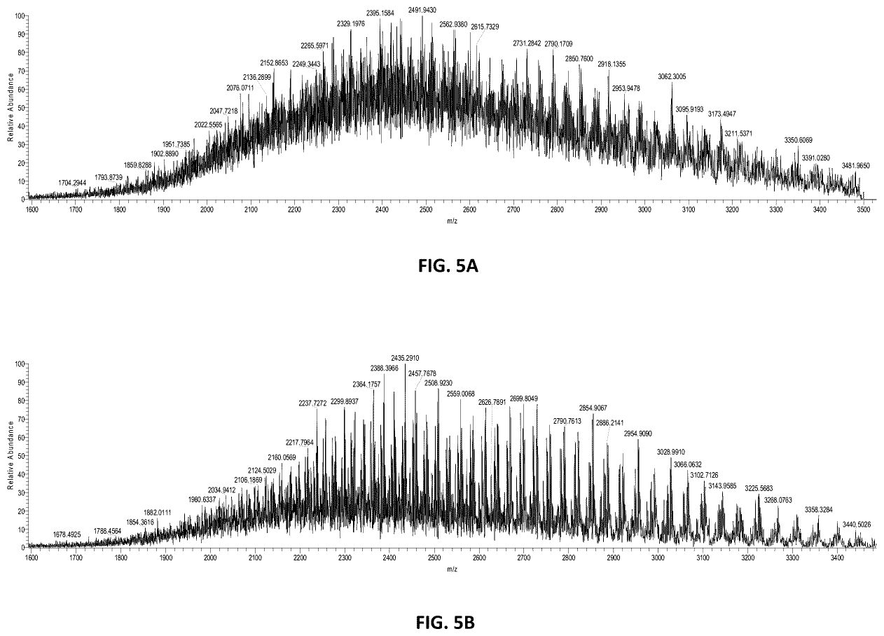

The difference in the homogeneity of bispecific antibodies containing SEQ ID NO: 6 and SEQ ID NO: 3 linkers was especially evident from the analysis of full-length BiXAb antibodies, which are symmetrical molecules that possess two linker sequences and thus possess an increased number of glycoforms in a BiXAb preparation. Additionally, BiXAbs possess two N-linked glycosylation sites, one on each Heavy Chain of the Fc-domain. The LC-MS spectra of BiXAb3a (constructed with the SEQ ID NO: 6 linker) and of BiXAb3b (constructed with the SEQ ID NO: 3 linker) are presented in FIGS. 5A and 5B, respectively. The BiXAb3a is expected to contain 8 additional O-linked glycosylation sites relative to the BiXAb3b, based on the differences in the linker sequences. The large number of O-linked glycosylation sites in BiXAb3a explains the highly complex MS spectrum in FIG. 5A. Both BiXAb antibodies possess 2 N-glycosylation sites in the Fc-domain resulting in additional glycoforms contributing to the heterogeneity of both BiXAbs, which explains the residual amount of heterogeneity in BiXAb3b observed in FIG. 5B.

1. A multispecific antigen-binding fragment comprising at least two Fab fragments, wherein each Fab fragment recognizes a different epitope of interest, and said Fab fragments are tandemly arranged in any order, the C-terminal end of the CH1 domain of a first Fab fragment being linked to the N-terminal end of the VH domain of the following Fab fragment through a polypeptide linker,
characterized in that the polypeptide linker sequence comprises or consists of amino acid sequence EPKX₁CDKX₂HX₃X₄PPX₅PAPELLGGPX₆X₇PPX₈PX₉PX₁₀GG (SEQ ID NO: 1), wherein X₁, X₂, X₃, X₄, X₅, X₆, X₇, X₈, X₉, X₁₀, identical or different, are any amino acid; with the proviso that the linker sequence does not comprise nor consist of sequences
(SEQ ID NO: 5)
EPKSCDKTHTCPPCPAPELLGGPSTPPTPSPSGG
or
(SEQ ID NO: 6)
EPKSCDKTHTSPPSPAPELLGGPSTPPTPSPSGG.

2. The multispecific antigen-binding fragment of claim 1, wherein:
a) X₁, X₂ and X₃, identical or different, are selected from the group consisting of Threonine (T), Serine (S), Alanine (A), Glycine (G), Valine (V), Asparagine (N), Aspartic acid (D) and Isoleucine (I);
b) X1 is Alanine (A);
c) X₂ and X₃, identical or different, are Threonine (T) or Serine (S);
d) X₄ and X₅, identical or different, are any amino acid selected from the group consisting of Serine (S), Cysteine (C), Alanine (A), and Glycine (G);
e) X₆, X₇, X₈, X₉, X₁₀, identical or different, are any amino acid other than Threonine (T) or Serine (S);
f) X₄ is Serine (S) or Cysteine (C);
g) X₅ is Alanine (A) or Cysteine (C); or
h) X₆, X₇, X₈, X₉, X₁₀, identical or different, are selected from the group consisting of Alanine (A), Glycine (G), Valine (V), Asparagine (N), Aspartic acid (D) and Isoleucine (I).

3. The multispecific antigen-binding fragment of claim 1, wherein:
X₁, X₂ and X₃, identical or different, are any amino acid;
X₄ and X₅, identical or different, are any amino acid selected from the group consisting of Serine (S), Cysteine (C), Alanine (A), and Glycine (G); and
X₆, X₇, X₈, X₉, X₁₀, identical or different, are selected from the group consisting of Alanine (A), Glycine (G), Valine (V), Asparagine (N), Aspartic acid (D) and Isoleucine (I).

4. The multispecific antigen-binding fragment of claim 3, wherein X₁, X₂ and X₃, identical or different, are selected from the group consisting of Threonine (T), Serine (S), Alanine (A), Glycine (G), Valine (V), Asparagine (N), Aspartic acid (D) and Isoleucine (I).

5. The multispecific antigen-binding fragment of claim 3, wherein X1 is Alanine (A).

6. The multispecific antigen-binding fragment of claim 3, wherein X₂ and X₃, identical or different, are Threonine (T) or Serine (S).

7. The multispecific antigen-binding fragment of claim 3, wherein:
a) X₄ is Serine (S) or Cysteine (C); or
b) X₅ is Alanine (A) or Cysteine (C).

8. The multispecific antigen-binding fragment of claim 3, wherein X₆, X₇, X₈, X₉, X₁₀, identical or different, are selected from the group consisting of Alanine (A) and Glycine (G).

9. The multispecific antigen-binding fragment of claim 3, wherein X₆ and X₇ are identical and are selected from the group consisting of Alanine (A) and Glycine (G).

10. The multispecific antigen-binding fragment claim 1, wherein the polypeptide linker sequence comprises or consists of a sequence selected from the group consisting of:
(SEQ ID NO: 3)
EPKSCDKTHTSPPAPAPELLGGPAAPPAPAPAGG;
(SEQ ID NO: 2)
EPKSCDKTHTSPPAPAPELLGGPGGPPGPGPGGG;
and
(SEQ ID NO: 4)
EPKSCDKTHTSPPAPAPELLGGPAAPPGPAPGGG.

11. The multispecific antigen-binding fragment of claim 1, which recognizes both EGFR and HER2/neu.

12. The multispecific antigen-binding fragment of claim 1, which recognizes both CD38 and PD-L1.

13. The multispecific antigen-binding fragment of claim 1, said multispecific antigen-binding fragment comprising at least two Fab fragments with different CH1 and CL domains.

14. A multispecific antibody having two identical antigen-binding arms, each consisting of a multispecific antigen-binding fragment of claim 1.

15. The multispecific antibody of claim 14, which has an immunoglobulin-like structure, comprising:
two identical antigen-binding arms each consisting of a multispecific antigen-binding fragment;
the dimerized CH2 and CH3 domains of an immunoglobulin;
the hinge region of an IgA, IgG, or IgD, linking the C-terminal ends of CH1 domains of the antigen-binding arms to the N-terminal ends of the CH2 domains.

16. A multispecific antibody comprising two heavy chains and four light chains,
wherein each heavy chain comprises:
a) a Fc region of an immunoglobulin comprising Hinge-CH2-CH3 domains,
b) which Fc region is linked to Fab heavy chain CH1-VH of antibody 1 (Ab1) by said Hinge domain,
c) which in turn is linked to Fab heavy chain CH1-VH of antibody 2 (Ab2), by a polypeptide linker sequence, wherein the polypeptide linker sequence links the N-terminus of said Fab heavy chain VH domain of Ab1 with the C-terminus of said CH1 domain of Ab2,
and the four light chains comprise light chains of Ab1 and light chains of Ab2 associated with their cognate heavy chain domains;
wherein the polypeptide linker sequence comprises or consists of amino acid sequence
(SEQ ID NO: 1)
EPKX₁CDKX₂HX₃X₄PPX₅PAPELLGGPX₆X-PPX₈PX₉PX₁₀GG,
wherein X₁, X₂, X₃, X₄, X₅, X₆, X₇, X₈, X₉, X₁₀, identical or different, are any amino acid; with the proviso that the linker sequence does not comprise nor consist of sequences
(SEQ ID NO: 5)
EPKSCDKTHTCPPCPAPELLGGPSTPPTPSPSGG
or
(SEQ ID NO: 6)
EPKSCDKTHTSPPSPAPELLGGPSTPPTPSPSGG.

17. The multispecific antibody of claim 16, wherein:
X₁, X₂ and X₃, identical or different, are any amino acid;
X₄ and X₅, identical or different, are any amino acid selected from the group consisting of Serine (S), Cysteine (C), Alanine (A), and Glycine (G); and
X₆, X₇, X₈, X₉, X₁₀, identical or different, are selected from the group consisting of Alanine (A), Glycine (G), Valine (V), Asparagine (N), Aspartic acid (D) and Isoleucine (I).

18. The multispecific antibody of claim 16, wherein X₁, X₂ and X₃, identical or different, are selected from the group consisting of Threonine (T), Serine (S), Alanine (A), Glycine (G), Valine (V), Asparagine (N), Aspartic acid (D) and Isoleucine (I).

19. The multispecific antibody of claim 16, wherein X₁ is Alanine (A).

20. The multispecific antibody of claim 16, wherein X₂ and X₃, identical or different, are Threonine (T) or Serine (S).

21. The multispecific antibody of claim 16, wherein:
a) X₄ is Serine (S) or Cysteine (C); or
b) X₅ is Alanine (A) or Cysteine (C).

22. The multispecific antibody of claim 16, wherein X₆, X₇, X₈, X₉, X₁₀, identical or different, are selected from the group consisting of Alanine (A) and Glycine (G).

23. The multispecific antibody of claim 16, wherein Ab1 and Ab2, being different, independently are selected from the group consisting of cetuximab or a mutated derivative thereof, on the one hand, and trastuzumab, or a mutated derivative thereof, on the other hand.

24. A polypeptide which comprises a heavy chain of the antigen-binding fragment or a heavy chain of the multispecific antibody as defined in claim 1.

25. A polynucleotide comprising a sequence encoding the polypeptide of claim 24.

26. A host cell transfected with an expression vector comprising the polynucleotide of claim 25.

27. The host cell of claim 26, further transformed with at least two polynucleotides encoding two different light chains: a first light chain pairing specifically with a first VH/CH1 region of said heavy chain; a second light chain pairing specifically with a second VH/CH1 region of said heavy chain.

28. The host cell of claim 27, which is additionally transformed with a third polynucleotide encoding a third light chain which is different from the first and second light chains, and which pairs specifically with a third VH/CH1 region of said heavy chain.

29. A method for preparing an antigen-binding fragment or a multispecific antibody comprising culturing a host cell of claim 26 and recovering said antigen-binding fragment or antibody from said culture.

30. A method of treating cancer comprising administering a multispecific antigen-binding fragment of claim 1 to a subject having cancer, wherein said multispecific antigen-binding fragment:
recognizes both EGFR and HER2/neu;
comprises cetuximab or a mutated derivative thereof, on the one hand, and trastuzumab, or a mutated derivative thereof, on the other hand; or
recognizes both CD38 and PD-L1.

31. A method of treating cancer comprising administering a multispecific antibody of claim 16 to a subject having cancer, wherein said multispecific antibody:
recognizes both EGFR and HER2/neu;
comprises cetuximab or a mutated derivative thereof, on the one hand, and trastuzumab, or a mutated derivative thereof, on the other hand; or
recognizes both CD38 and PD-L1.

32. A polypeptide which comprises
(SEQ ID NO: 1)
EPKX₁CDKX₂HX₃X₄PPX₅PAPELLGGPX₆X₇-PPX₈PX₉PX₁₀GG,
wherein X₁, X₂, X₃, X₄, X₅, X₆, X₇, X₈, X₉, X₁₀, identical or different, are any amino acid as defined in claim 1; with the proviso that the polypeptide does not comprise
(SEQ ID NO: 5)
EPKSCDKTHTCPPCPAPELLGGPSTPPTPSPSGG
or
(SEQ ID NO: 6)
EPKSCDKTHTSPPSPAPELLGGPSTPPTPSPSGG.

33. The polypeptide of claim 32, which sequence is selected from the group consisting of:
(SEQ ID NO: 3)
EPKSCDKTHTSPPAPAPELLGGPAAPPAPAPAGG;
(SEQ ID NO: 2)
EPKSCDKTHTSPPAPAPELLGGPGGPPGPGPGGG;
and
(SEQ ID NO: 4)
EPKSCDKTHTSPPAPAPELLGGPAAPPGPAPGGG.


SEQUENCE LISTING (23 sequences)

SEQ ID NO: 1 - protein, 34 aa, synthetic construct
  REGION 1..34: note = linker
  SITE 4: note = misc_feature - Xaa can be any naturally occurring amino acid
  SITE 8: note = misc_feature - Xaa can be any naturally occurring amino acid
  REGION 10..11: note = misc_feature - Xaa can be any naturally occurring amino acid
  SITE 14: note = misc_feature - Xaa can be any naturally occurring amino acid
  REGION 24..25: note = misc_feature - Xaa can be any naturally occurring amino acid
  SITE 28: note = misc_feature - Xaa can be any naturally occurring amino acid
  SITE 30: note = misc_feature - Xaa can be any naturally occurring amino acid
  SITE 32: note = misc_feature - Xaa can be any naturally occurring amino acid
EPKXCDKXHXXPPXPAPELLGGPXXPPXPXPXGG

SEQ ID NO: 2 - protein, 34 aa, synthetic construct
  REGION 1..34: note = linker
EPKSCDKTHTSPPAPAPELLGGPGGPPGPGPGGG

SEQ ID NO: 3 - protein, 34 aa, synthetic construct
  REGION 1..34: note = linker
EPKSCDKTHTSPPAPAPELLGGPAAPPAPAPAGG

SEQ ID NO: 4 - protein, 34 aa, synthetic construct
  REGION 1..34: note = linker
EPKSCDKTHTSPPAPAPELLGGPAAPPGPAPGGG

SEQ ID NO: 5 - protein, 34 aa, synthetic construct
  REGION 1..34: note = linker
EPKSCDKTHTCPPCPAPELLGGPSTPPTPSPSGG

SEQ ID NO: 6 - protein, 34 aa, synthetic construct
  REGION 1..34: note = linker
EPKSCDKTHTSPPSPAPELLGGPSTPPTPSPSGG

SEQ ID NO: 7 - protein, 120 aa, Homo sapiens [sequence omitted]

SEQ ID NO: 8 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 9 - protein, 119 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 10 - protein, 98 aa, Homo sapiens
ASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKV

SEQ ID NO: 11 - protein, 15 aa, Homo sapiens
EPKSCDKTHTCPPCP

SEQ ID NO: 12 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 13 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 701 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPAPAPELLGGPGGPPGPGPGGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 214 aa, Homo sapiens
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 122 aa, Homo sapiens [sequence omitted]

SEQ ID NO: 18 - protein, 702 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 214 aa, Homo sapiens
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 475 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 22 - protein, 10 aa, Homo sapiens
PSTPPTPSPS

SEQ ID NO: 23 - protein, 118 aa, Homo sapiens [sequence omitted]